(19) United States
(12) Patent Application Publication
(10) Pub. No.: US 2005/0032092 A1
(43) Pub. Date: Feb. 10, 2005

(54) Use of metal oxide semiconductors to manipulate biological molecules

(76) Inventors: Tijana Rajh, Naperville, IL (US); Tatjana Paunesku, Chicago, IL (US); Gayle E. Woloschak, Chicago, IL (US); Marion C. Thurnauer, Downers Grove, IL (US)

Correspondence Address: CHERSKOV & FLAYNIK;The Civic Opera Building, Suite 1447, 20 North Wacker Drive, Chicago, IL, 60606, US

(21) Appl. No.: 10/823,509

(22) Filed: Apr. 12, 2004

Related U.S. Application Data
(63) Continuation-in-part of application No. 10/755,045, filed on Jan. 9, 2004.
Application No. 10/755,045 is a continuation of application No. 09/606,429, filed on Jun. 28, 2000, now Pat. No. 6,677,606.

(51) Int. Cl. C12Q 1/68; G01N 33/53; H01L 35/24; H01L 51/00

(52) U.S. Cl. 435/6; 435/7.1; 205/777.5; 257/40

(57) ABSTRACT

A method is provided for selective binding and detecting target molecules, and a method for detecting biological molecules, the method comprising supplying a semi-conductor capable of charge pair separation, and juxtaposing affinity moieties to the semi-conductor so as to effect changes in the charge pair separation characteristics when the affinity molecules are bound to the target molecules. Also provided is a construct to facilitate in vivo and in situ manipulation of biological material such as DNA, RNA, organelles, and protein. A method also is provided to facilitate in vivo and in situ manipulation of biological material.

This patent application is a Continuation in Part of U.S. patent application Ser. No. 10/755;045 filed Jan. 9, 2004, which was a Continuation application of U.S. patent application Ser. No. 09/606,429, now U.S. Pat. No. 6,677,606 B1 which issued on Jan. 13, 2004.

### CONTRACTUAL ORIGIN OF THE INVENTION

The United States Government has rights in this invention pursuant to Contract Number W-31-109-ENG-38 between the United States Government and Argonne National Laboratory.

### BACKGROUND OF THE INVENTION

1. Field of the Invention

This invention relates to a method for attaching and detecting the attachment of biological molecules to semiconductors, and more particularly, the invention relates to a method for attaching biologically active molecules to nanoparticle-size metal oxide semi-conductors. The invention also relates to the use of semiconductors to manipulate biological molecules in vivo and in situ.

2. Background of the Invention

Detection of target molecules in an unknown mixture finds a variety of applications. A few of these applications include genome sequencing, forensics, assays, and drug affinity studies.

Typical detection techniques involve the use of fluorescence tags. Such tags are first attached to moieties (having known affinities to target molecules), to create a construct. The construct is then combined with materials in a search for the target molecules suspected of residing in those materials.

A myriad of problems exist with the use of fluorescence molecules in detection schemes. For example, the tags tend to fluoresce at a wide wavelength band and therefore obliterate the “fingerprint” of other concomitantly used fluorescent tags.

Also, fluorescence moieties are short-lived, particularly at wavelengths required to induce fluorescence. As such, exposure times to the wavelengths must often be minimized. Strict ambient conditions also are required to forestall eventual tag denigration.

Efforts have been made to eliminate fluorescence tag usage in detection processes. For example, U.S. Pat. No. 5,990,479 awarded to Weiss et al on Nov. 23, 1999, supplants fluorescence moieties with semiconductor moieties. The semiconductors are attached to affinity molecules to create a construct which in turn is mixed with material suspected of containing target molecules. Detection is noted when the mixture is subjected to light at wavelengths which cause the semi-conductor in the construct to luminesce. A problem with fluorescent tags is that each dye requires a different excitation energy. The tags also are unstable when subjected to illumination.

The '479 patent eliminates many of the drawbacks of some fluorescence systems. For example, each semiconductor imparts luminescence at narrow band wavelengths. This feature allows several semiconductors, each with characteristic emission spectra, to be used simultaneously to detect several different target molecules.

However, state of the art semiconductor detection systems do not provide a means for determining the amount of target moiety detected. Also, detection sensitivities are limited to optical characteristics of the semiconductor.

Notwithstanding the foregoing drawbacks in semiconductor systems, the inventors envision exploiting the phenomenon in those substrates whereby radical intermediates are formed following light induced charge pair formation. Electron Paramagnetic Resonance (EPR) is the prime technique for detecting these formations.

Nanocrystalline metal oxide semiconductor particles that are durable and are not susceptible to photo-degradation, act as miniaturized electrochemical cells and act as stable and efficient artificial photosynthetic systems. However, the recombination kinetics in these systems is very fast, on the order of picoseconds. N. Serpone et al. J. Phys. Chem., 99, 16655 (1995). Unless the charge separation is increased by reaction with adsorbed species, the efficiency of charge separation is very low.

A new wave of functional genomics is on the horizon to take advantage of the information provided by the human genome initiative. Functional genomics relies at least somewhat on “interference techniques” (such as siRNAs) to dissect signal transduction pathways.

Interference with gene expression (antisense therapies) in vivo is also used medically. However, gene therapy (viewed as both the transfer, disabling, enabling and/or the removal of genes from cells with therapeutic intent) promises a permanent solution for dealing with genes expressed at the wrong time or in the wrong place. Currently, however, gene therapy depends on brute-force approaches to cell entry and low-efficiency intracellular reactions carried out in the presence of low local concentrations of reactants. D. Kerr “Clinical Development of Gene Therapy for Colorectal Cancer” Nat. Rev Cancer 2003, August; 3(8), pp 615-22. C. K. Kim et al “Gene Medicine: A New Field of Molecular Medicine” Arch Pharm Res. 2001, February; 24(1) pp 1-15.

Research has been conducted whereby in situ gene correction is attempted via the use of ribozymes to alter RNA and DNA expression. M. B. Long et al., “Ribozyme-mediated Revision of RNA and DNA”, J. Clin Invest. 2003 August: 112(3):312-8. However, this method often results in the DNA remaining intact, so that the therapy has to be reapplied to the target cells.

Also, multifunctional nanorods have been employed in attempts to facilitate intracellular DNA delivery. A. K. Salem, et al. “Multifunctional Nanorods for Gene Delivery”, Nat Matter 2 (10), pp 668-671 (2003 Sep. 14). However, at best, these structures only aid in DNA delivery. No solutions to in situ manipulation is offered.

A need exists in the art for a detection system based on electronic changes in a foundation substrate. The system should incorporate a means to modify the charge separation tendencies of photo-induced ion pairs on the substrates so as to be measurable with existing time-resolved detection systems. This modification would allow chemical reactions to be efficiently performed using nanocrystalline materials. The system should also serve as a detector for the existence of moieties that would modify the charge separation fingerprint via electron donation or extraction.

A need also exists for a construct and method for manipulating biological entities, such as DNA, RNA, protein, and organelles in vivo and in situ. The construct and method should be easily introduced into the cell or organism. Also, the mechanism for initiating manipulation should be remotely controlled.

### SUMMARY OF THE INVENTION

An object of the present invention is to provide a method for detecting molecules that overcomes many of the disadvantages of the prior art.

Another object of the present invention is to provide a method for exploiting the charge separation abilities of semi-conductors to create detectors of molecules. A feature of the invention is the utilization of a bidentate or tridentate modifier molecule as an electron donor or acceptor to the semi-conductor. An advantage of the invention is the prolongation of charge separation on the semi-conductor as an indicator of the type of molecule juxtaposed and electrically connected to the semi-conductor particle.

Yet another object of the present invention is to provide a method for selective binding and modifying of molecules in vivo or in vitro. A feature of the method is that a nanocrystalline-biological construct, capable of photochemical response, and capable of simultaneously carrying a number of biologically active molecules, can bypass biological membranes via standard delivery methods. An advantage of the method is that oxidative damage, produced by positive charge centers (resulting in the formation of oxygen centered radicals covalently linked to surface semi-conductor atoms), facilitates the cleaving of particle-attached molecules to reactive sites. Another advantage is that a collection of biologically active molecules can be delivered to, and therefore co-localized at, the reactive sites to facilitate simultaneous action of the delivered biomolecules.

Still another object of the present invention is to provide a molecule detection system which also quantifies the amount of target molecule present. A feature of the present invention is the utilization of a nanocrystalline foundation material capable of binding a plurality of linker moieties, whereby the moieties link the material to the target molecule. An advantage of the invention is that the detector construct facilitates selective adsorption and selective chemical reactions at the surface of the material.

Another object of the present invention is to provide a radiation-inducible nucleic acid endonuclease. A feature of the invention is that a semiconductor particle, when in electrical communication with a biological moiety such as DNA or RNA, and when subjected to electromagnetic waves of a predetermined energy, extracts electrons from the biological moiety leaving electropositive holes in the moiety, thereby inducing cleavage in the moiety. An advantage of the invention is that the biological moiety, and/or any covalently attached biological structure, can be manipulated in vivo and in situ while other similar molecules in solution remain intact.

Yet another object of the present invention is to provide a method for manipulating biological mechanisms in vivo and in situ. A feature of the invention is the use of semi-conductor containing constructs to deliver voltages to target genome sequences. An advantage of the invention is the selective application of voltage to the target sequences when radiation is directed to the construct.

Another object of the invention is to provide a sequence specific endonuclease. A feature of the invention is a construct which facilitates hole transfer across a nanoparticle-biological molecule interface when electromagnetic radiation greater than 1.6 eV contacts the construct. An advantage of the invention is that an accumulation of holes in the biological molecule changes the molecule at a point determinable from the location of the holes.

Briefly, the invention provides for a method for detecting molecules, the method comprising determining the electronic status of a semi-conductor; establishing electronic communication between the molecules and the semiconductor; subjecting the semi-conductor to energy influx; and redetermining the electronic status of the semi-conductor.

Also provided is a method for detecting biological molecules, the method comprising supplying a semi-conductor having a first energy level and a second energy level and whereby the first energy level corresponds to a first optical characteristic of the semi-conductor; establishing electrical contact between the semi-conductor and the molecules; causing electrons to move from the molecules to the second energy level; and monitoring any change in the first optical characteristic.

Also provided is a method for detecting target moieties in situ, the method comprising binding biological material to nanocrystalline semiconductor particles, wherein the material has an affinity to the target moiety; facilitating entry of the bound material into an organelle; and subjecting the semiconductor to radiation sufficient to produce a charge pair separation on the semiconductors surface.

The invention further provides a method to manipulate biological material in vivo and in situ, the method comprising supplying a construct further comprising a semi-conductor and a biological moiety, inserting the construct into a target area in a living organism, and creating a plurality of charges on the construct wherein the size of the charges and distances between the charges cause the target area to change in physical structure.

### BRIEF DESCRIPTION OF THE DRAWING

Other objects, features, and advantages of the present invention will become apparent from the following description and the accompanying drawings, wherein:

FIG. 1 is a schematic diagram of the electronic state of semiconductor particles, in accordance with features of the present invention;

FIG. 2 is a schematic diagram of the fate of photogenerated charge pairs on particulate semi-conductors, in accordance with features of the present invention;

FIG. 3 is a reaction sequence depicting the formation of the invented semi-conductor-biological molecule construct, in accordance with features of the present invention;

FIG. 4 is a graph showing a red-shift of modified nanocrystalline particles, in accordance with features of the present invention;

FIG. 5 is a schematic diagram showing an electron cascade from a molecule toward the modified surface of a semi-conductor particle, in accordance with features of the present invention;

FIG. 6 is a photomicrograph of a nucleus of a cell showing the presence of the invented particle-biological construct, in accordance with features of the present invention;

FIG. 7 is a photograph of gel electrophoresis depicting light-induced cleavage of biological molecules from nanoparticles, in accordance with features of the present invention.

FIG. 8 is another depiction of gel electrophoresis depicting light-induced cleavage of biological molecules, in accordance with features of the present invention, and

FIG. 9 is a depiction of gel electrophoresis depicting radio-label-induced cleavage of biological molecules, in accordance with features of the present invention.

### DETAILED DESCRIPTION OF THE INVENTION

The invention relates to multiple biological use of nanocrystalline semiconductors bound to oligonucleotides, DNA segments and peptides for selective binding and hybridization, photophysical and photochemical DNA and protein chemistry in vitro and in vivo. In one specific example, surface modification of nanocrystalline metal oxide particles with ortho substituted hydroxylated aromatic ligands was found to result in bidentate coordination of surface Ti atoms. Due to the specific bindings of surface modifiers, the optical properties of small titania particles change and the onset of absorption shifts to the red, compared to unmodified nanocrystallites (for salicylate 0.8 eV, ascorbate 1.6 eV and dopamine 1.85 eV). The binding is exclusively characteristic of small particle colloids in the nanocrystalline domain and was found to be a consequence of adsorption induced reconstruction of nanoparticle surface.

A cornerstone of the invention is the enhanced charge separation and improved optical properties of nanocrystalline semi-conductors that involve photoinduced interfacial electron transfer from surface modifiers into one or more regions of the semi-conductors. The charge pairs are instantaneously separated into two phases, the holes on the donating organic modifier and the donated electrons in either the conduction band or valence band of the semiconductor. In some instances, this charge separation exceeds 80 angstoms (Å). In instances where the semiconductor is attached to the dopamine, the distance can be up to 100 Å in guanine-depleted sequences. Positive hole trapping ultimately occurs in the sugar moiety of the phosphodiester backbone up to 20 nucleotides away from the semiconductor particle. The localization of hole trapping leads to cleavage at that point.

The inventors have found that the introduction of interspersed guanine moiety facilitates long range hole transport. Long distance charge transport through guanines allows hole hopping and in the guanine rich sequences, charge transport distances increase by 400 percent compared to when single occurrences of guanines are present. In guanine-depleted sequences attached to TiO₂-constructs, a photogenerated hole will travel for 80 A to find a guanine base. If a plurality of guanine bases are interspersed throughout the DNA, the hole will travel even further by passing over single guanine sites to reach deeper trapping sites such as GG, GGG, etc. This passing over or hole extension can occur up to approximately 400 Å. As such, this transference allows oxidative cleavage at large distances from the nanoparticle surface, (again, up to approximately 400 Å).

Trapping initially occurs at the sugar-base interfaces, (for example at the guanosines or uridines), and typically at sites having multiple such interfaces. Guanosine has the most favorable thermodynamic properties, i.e., the least positive redox potential of all the nucleotides. Strand cleavage occurs by radical reorganization and breaking of the phosphodiester bond in the DNA backbone adjacent to guanosine trapping sites.

The inventors have found that the enhanced charge build up caused by the presence of the semiconductor particle overwhelms the capacity of nucleic acid molecules to self repair. For example, normally the rates of reactions leading to DNA cleavage (i.e., the reaction of guanine cation radial with water) is slower than charge recombination in contact ion pairs. This protects the DNA from photochemical damage. However, the charge accumulation caused by the invented nanoparticle-biological matter construct leads to repeated hole creation and accumulation on the biological molecule attached to the semiconductor.

By inducing the charge separation in vivo and in situ, the inventors have induced cleavage of a first set of nucleic acid molecules attached to the semiconductor particle, and even to a second set of nucleic acid molecules hybridized to the first set. The invented construct can manipulate a single instance of a single sequence in a cell. As mentioned supra, this manipulation occurs when the construct is exposed to radiation. This radiation inducibility confers the in vivo and in situ manipulation capability of the construct.

In one embodiment, the inventors found that TiO₂ nanocomposites are capable of cleavage of the attached DNA or its complementary strand upon excitation with energies as low as 1.6 eV (such as that coming from white light, UV, X-rays or gamma rays, alpha rays, beta rays.). Radioactive particles (i.e. probes such as phosphorus-32, iodine-131, sulfur 35, selenium 75, technetium-99 and yttrium-90) attached to the nanoparticle enhances the semi-conductor cross section of interaction, enabling the process to occur at lower eV values than those supplied by typical x-rays or gamma rays. Cleavage typically occurs along the phosphodiester backbone but can also occur along the base-pair midline. Generally, probes with short half-lives are utilized so that the construct will deactivate itself upon depletion of radiation emissions.

Surface modifications of the nanocrystalline particle with bidentate ortho-substituted hydroxylated electron donating ligands, combined with laser excitation of the modified particle, causes separation resulting in correlated radical pair electron spin polarization mechanisms, reminiscent of the electron cascade seen in optimized natural photosynthetic systems.

The invention exploits the electrochemical cell characteristics of particulate semiconductors. It should be noted that while a myriad of semiconductors can be utilized, titania is depicted herein for the sake of illustration.

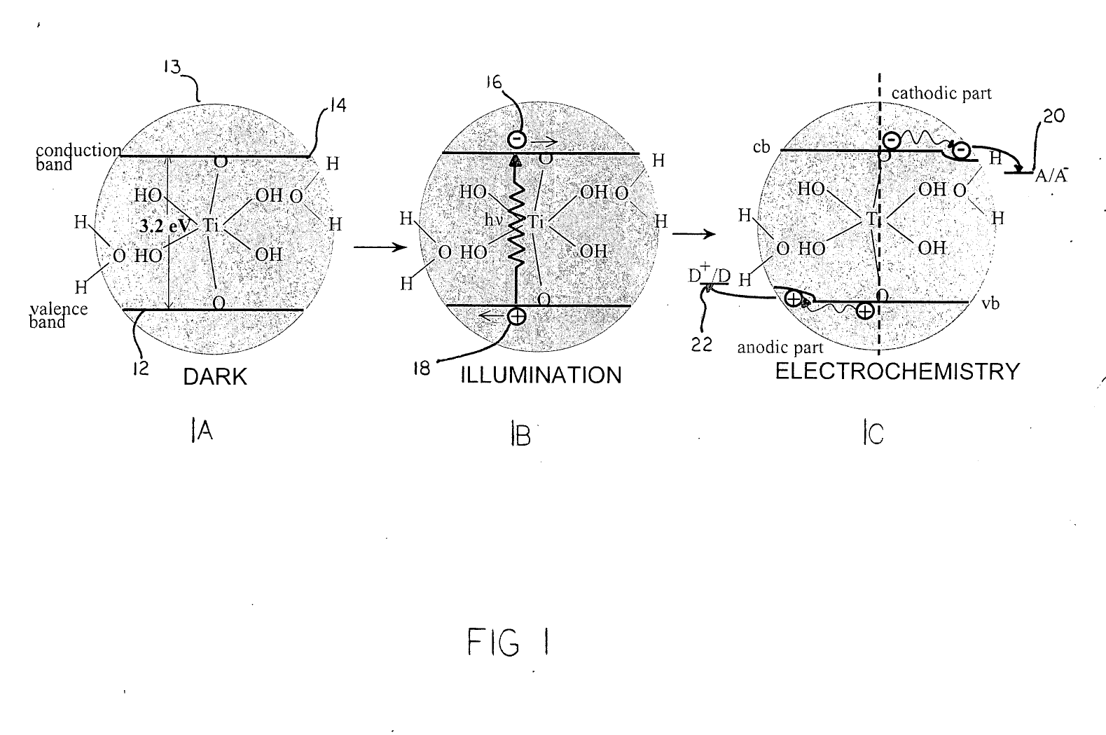

As depicted in FIG. 1A, semiconductors have an energy band structure characterized by a gap between the highest occupied energy level 12 (or valence band) and the lowest unoccupied energy level 14 (or conduction band). In the case of titanium oxide, the gap is 3.2 eV. A single semiconductor particle 13 is depicted. The inventors have found that when the semiconductor particles are associated with dopamine, the band gap shifts to lower energies, of approximately 1.6 eV. This lower energy value enables the construct's use in vivo.

A disturbance in the energy level of the electrons in the valence band in the semiconductor is induced via illumination (FIG. 1B). Upon illumination with photons having energy greater than the band gap, an electron 16 is excited to the conduction band 14 while in the valence band 12 a positive 18 hole is created.

As depicted in FIG. 1C, the electrons and holes generated via the illumination can separate and diffuse to the surface of the semiconductor. This surface diffusion allows the diffused electrons and holes to react with redox couples to undergo reduction reactions 20 and oxidation reactions 22, respectively. Specifically, these photogenerated electrons and holes migrate to the particle/solute interface (the solute being a moiety adjacent to the particle and perhaps covalently bound thereto) and/or to the attached biomolecule where they can either recombine or react with redox species in solution. Such redox species in vivo and/or in situ include hybridized nucleic acid, nuclear DNA, mitochondrial DNA, RNA, proteins, receptors, membranes, or combinations thereof.

An exemplary semiconductor for use in the invented construct is titanium dioxide (TiO₂).

A cornerstone of the invention is the juxtaposition of redox moieties to the particle. Without the utilization of such close proximity electrophillic and nucleophillic moieties, rapid recombination of the photogenerated charge pairs (i.e. the electron 16 in the conductance band and the “hole” 18 in the valence band) occurs, particularly since the pairs are located on the same particle. As such, the inventors have devised machinery to convert light energy into chemical energy via a sequential electron transfer process whereby a series of electron trapping sites are utilized. The machinery serves to facilitate long distance charge separation by blocking the surface states of titania. This enhances the kinetics of desired reactions.

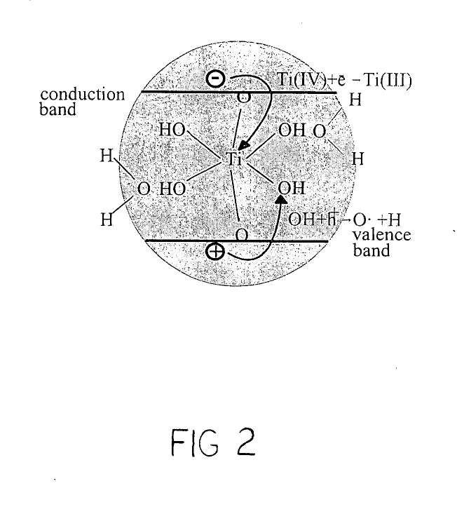

Electron Paramagnetic Resonance (EPR) has shown that electrons which have been energized to the conduction band, are trapped as reduced metal centers; in the case of TiO₂ the electrons facilitate reduction of Ti (IV) to Ti (III). Concomitantly, the holes left in the valence band are trapped as oxygen centered radicals covalently linked to surface titanium atoms. A schematic diagram of this phenomenon is provided as FIG. 2.

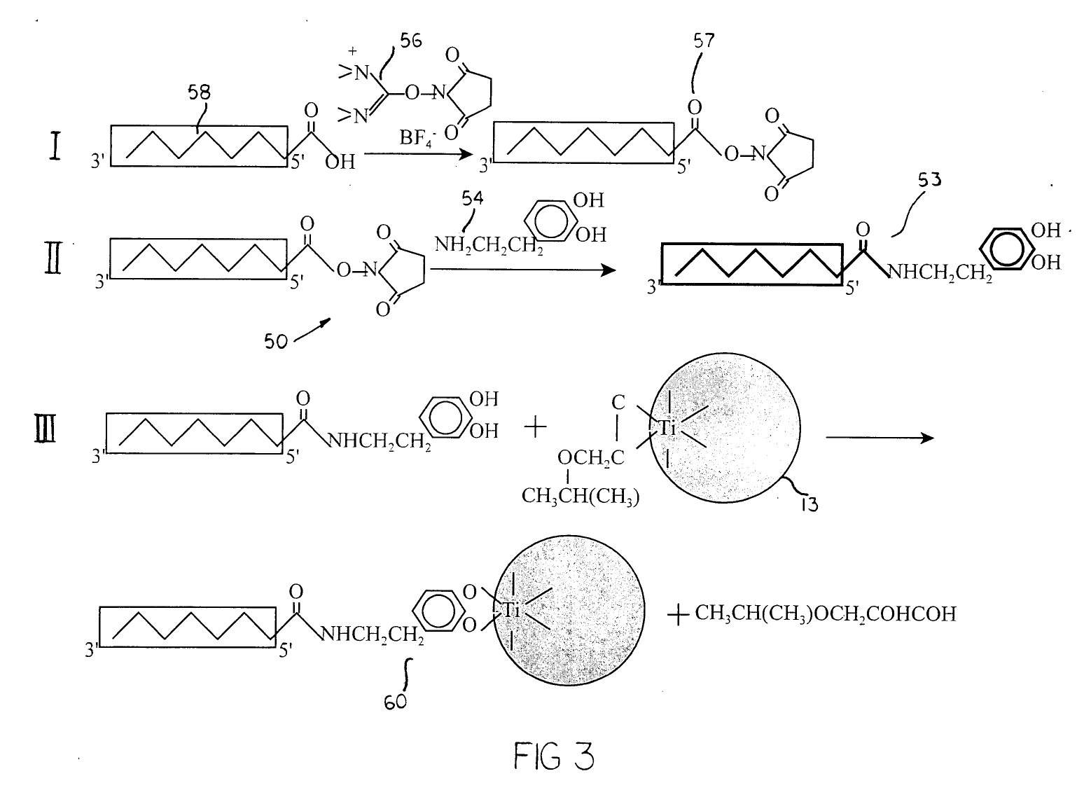

A particle-biological molecule construct has been invented, and is the product of an electrophilic reaction between the carboxylic terminus of oligonucleotide and amino-containing electrophilic reagents, the reaction depicted generally as numeral 50 in FIG. 3. Alternatively, the reaction can occur between the amino-containing terminus of an oligonucleotide and the carboxylic terminus of electrophilic reagents.

In the illustrated embodiment, a semiconductor particle (preferably one having octahedral structure, such as TiO₂) 13 is bound to oligonculeotide(s) 58 via a charge-transfer intermediary. This is accomplished by a series of electrophilic and nucleophillic substitutions.

In a first step, depicted as Roman Numeral I in FIG. 3, the oligonucleotide 58 is modified at its carboxylic terminus to contain an ester-moiety. First, the carboxyl terminus is transformed into a succinimide intermediate via extraction of the hydroxyl moiety. Then, in a nucleophillic substitution, the succinimide group is expelled and its place taken by a basic amino group 56 (a suitable amino group found on N-hydroxy-succinimide, as shown) to form an oligo nucleotide with an ester terminus 57.

It is this ester terminus which reacts with a modified oligonucleotide containing bidentate or tridentate modifiers 54 in reaction sequence II of FIG. 3, to arrive at the oligo-bidentate construct 60. One suitable class of modifiers are the 1,2 dihydroxyl phenyls, an exemplary species being dopamine. Generally, when oligonucleotides or PNAs are to be ultimately connected to the nanocrystalline particles, the intermediate modifier particles contain amino moieties. The modifiers are strongly coupled to the surface of the nanocrystalline titanium dioxide particles 52.

Additionally, titanium dioxide nanoparticles are bound to proteins, peptide nucleic acids (PNAs) or oligonucleotides. Generally, the particles are coupled to bidentate modified benzoic acids such as dihydroxyl benzoic acids (including but not limited to 1,2 dihydroxyl phenyl acetic acid), when proteins ultimately are to be complexed with the particles via an intermediate N-hydroxy-succinimide ester in a similar reaction sequence as that depicted in FIG. 3.

The remaining particle surface is protected to prevent undesirable reactions of hydroxyl groups at the titanium surface with carboxyl and phosphor-diester groups on the oligo. An exemplary means for protecting the surface is a layer of glycidyl isopropyl ether on the semi-conductors surface. Generally, any material which removes or otherwise blocks hydroxyl moieties on the titania surface from reacting with target molecules is suitable. In the case of the glycidyl isopropyl ether, a layer approximating 5 angstroms (Å) in thickness is suitable.

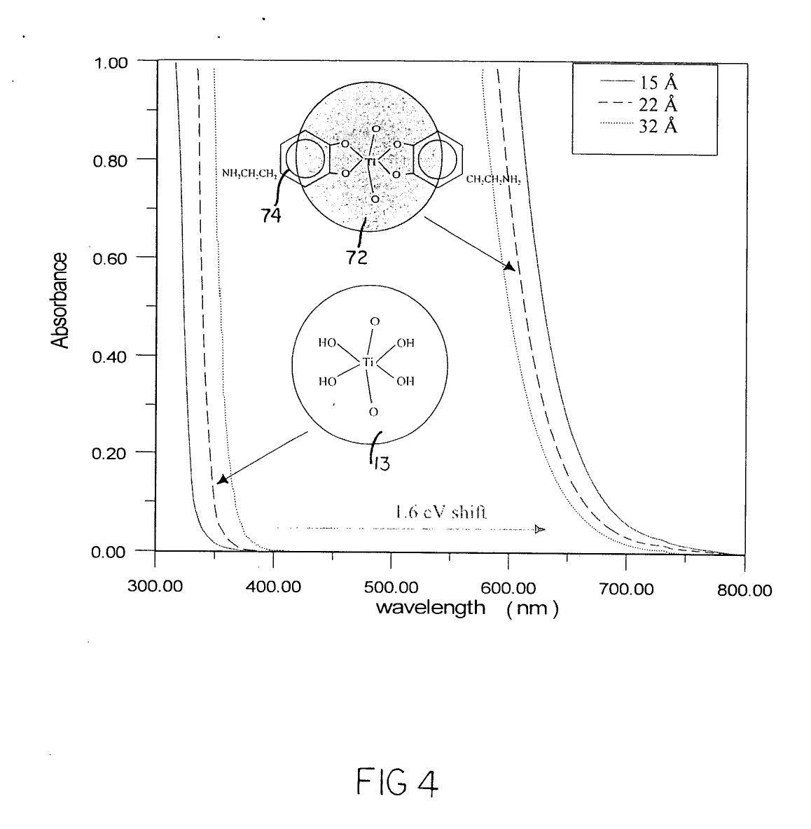

A salient feature of the invented process is the observation of a red-shift which results when certain modifiers are present on nano-sized semi-conductor particles. Specifically, and as depicted in FIG. 4, unmodified TiO₂ particles 70 exhibit absorbency at approximately 400 nm. However, when modifier moieties (such as dopamine, 74) are attached to the particle, the resulting construct 72 exhibits a shift in absorbency to an absorbence threshold of 760 nm.

The absorption in the red region reflects the presence of the excess electrons in the excited state of TiO₂ and is due to the absorption of localized and delocalized electrons in TiO₂. The inventors found that the injection of excess electrons in TiO₂, which has been modified with dopamine, will form an additional abhorrence above the shifted absorption threshold of 760 nm. The shape of this part of the spectrum in which the absorption spectrum of excess electrons shows characteristic increased absorption with longer wavelengths is consistent either with free carrier absorption and/or absorption to shallow defect sites on interstitial Ti atoms.

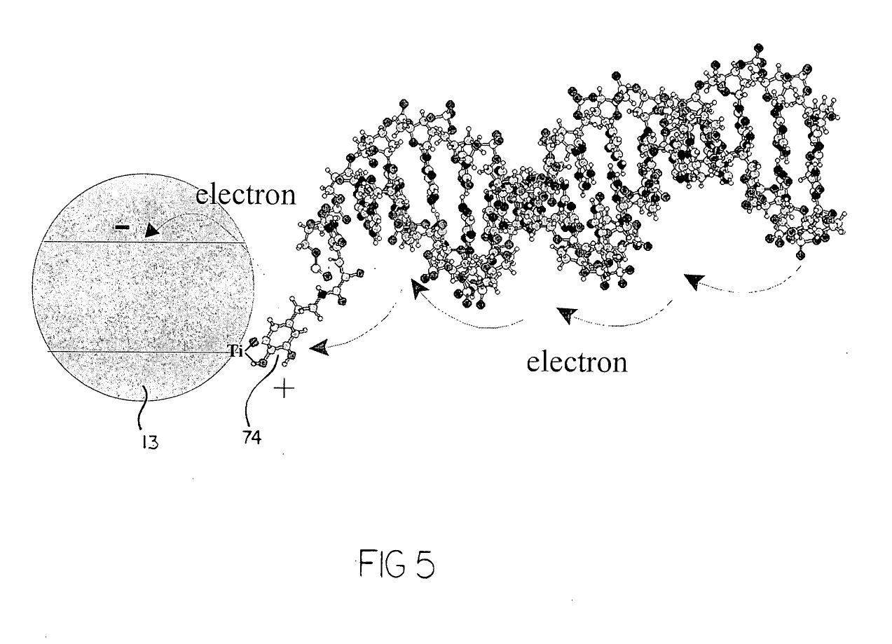

Degree of upshift serves as an indicator as to how much of an electron donor molecule is present and electronically communicating with the semi-conductor surface modifying agent, or the particle surface itself. For example, the inventors have found that a four-member ring results in less of a shift than a five-member electron donor. This is primarily due to the easier ability for resonance electrons to leave the larger ring structure and cascade toward the particle surface. A schematic depiction of such a cascading phenomenon is illustrated in FIG. 5 wherein DNA is shown facilitating sequential electron transfer to the surface of the particle.

Component

Detail

The illustrations and protocol provided herein utilize titanium oxide as the semi-conductor particle, solely for illustrative purposes. A myriad of metal oxides are suitable particle candidates. As such, aside from the titanium dioxide, other nanocrystalline (1-20 nm) octahedral metal oxides such as VO₂, ZrO₂, Fe₃O₄, Fe₂O₃, ZnO, MnO₂, NiO and CuO could be used.

As mentioned supra, it is preferable that the semi-conductors utilized be of an octahedral structure when dopamine or some similar dihydroxyl phenyl molecule is utilized as a surface modifier. Octahedral structure of the foundation nanocrystalline material is necessary in the illustrated construct so as to facilitate relatively unhindered coupling with the bond angles provided by the dopamine bridging group.

Generally though, any nanocrystalline-material and modifying surface molecule, which when combined maintain their respective surface geometries, are suitable components of a redox pair.

Regarding semi-conductor surface modifiers, a myriad of multifunctional ligands are suitable. Such ligands should have groups with varying donor acceptor character, selective binding of reactants, and for the binding to the surface of the semi-conductor particle. Preferably, suitable molecules used for the surface derivatization of the semiconductor particle will bind a wide selection of organic, biologically significant molecules, such as DNA, RNA, protein, membranes, organelles, receptors and vesicles. The following is a list of suitable bidentate and tridentate functional moieties:
mercapto (SH) amino (—NH₂) α-hydroxyl (—OH—OH) carboxyl (—COOH) phosphono (P(O)(OH)₂)

These moieties can be presented in different combinations, relative positions and hydrocarbon chain lengths. As noted supra, the choice of modifier molecules also will be dependant upon the bond angle geometry of the foundation nanocrystalline semiconductor material.

In one embodiment, the invention provides working solutions of modified semiconductor nanocrystallites (1-20 nm) which are modified with specific moieties. The modifier, such as oligonucleotide, provides both hole scavenging (photochemical and photophysical response) and selective binding functions in vitro and in vivo. Inasmuch as the oligonucleotide-dopamine-nanocrystalline construct is unobtrusive (1-20 nm when TiO2 is utilized), its insertion into biological systems (in vivo and in vitro) is not problematic.

Transfection

Detail

Transfection of eukaryotic cells with nanoparticle-oligonucleotide complexes were performed. Two different human cell lines (HL60 and HeLa) were transfected with six different oligonucleotides (4S, 2S, r18RS, r28RS, MIT1, and MIT2) bound to nanoparticles, wherein the oligonucleotides have the following base sequences:

| | |
|---|---|
| 2S | 5′ carboxy DT-GCATGCATGGATGGA |
| 4S | 5′ carboxy DT-GGATGGATGGATGGA |
| MIT1-S5T | 5′ carboxy DT-CCACTTTCCACACAG |
| MIT2-S5T | 5′ carboxy DT-AGACCAAGAGCCTTC |
| R18S-S5T | 5′ carboxy DT-TTCCTTGGATGTGGT |
| R28S-S5T | 5′ carboxy DT-CAGGATTCCCTCAGT |

Transfection was done with QIAGEN SuperFect Transfection Reagent (as follows (basically according to the manufacturer's instructions):

For HeLa cells, 200,000 cells each were plated on 60 mm 2 dishes a day before transfection. For HL60 cells 2,000,000 cells were plated on 60 mm² dishes on the day of transfection in 4 ml of cell growth medium (DMEM for HeLa and RPMI160 for HL60 cells) with 10% fetal calf serum and incubated at 37° C. with 5% CO₂.

Oligonucleotide-nanoparticle suspensions had 2 to 8 μM concentrations. Between 2 and 50 μl of solution was mixed with 20 μl of SuperFect Reagent and some serum free medium up to the final volume of 150 μl.

Mixtures were incubated at room temperature for 10 minutes, mixed with 1 ml of cell growth medium with serum and then applied on cells drop-wise. Cells were incubated 2 to 8 hours at 37° C. and 5% CO₂.

Cells were collected (HeLa cells were detached from the plate by trypsinization) by centrifugation of 10 minutes at 700 g, resuspended in Phosphate Buffered Saline (PBS), and re-precipitated. Finally, cells were resuspended in 40 μl of PBS and applied on EM grids. Cells prepared like this were dried by 10 min incubation in ethanol (100%) and Ti was detected by fluorescent microprobe at the Advanced Photon Source 2ID-E beamline at Argonne National Laboratory, Argonne, Ill.

A method for manipulating a genome with the invented construct is found in Paunesku, T. et al., Nature Materials 2, 343-346 (Apr. 13, 2003), and incorporated herein by reference.

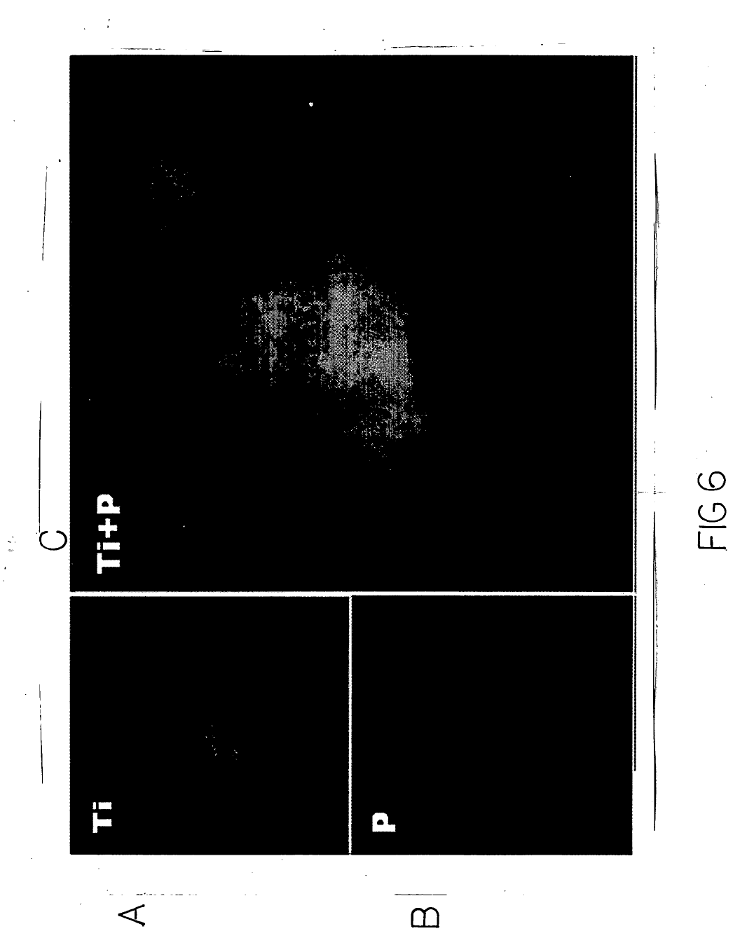

FIG. 6 depicts evidence of the construct residing in a nucleolus of a mammalian cell. PC12 cells were transfected with R18Ss-TiO₂ nanocomposite, and a free R18S as oligonucleotide. The R18Ss-TiO₂ oligonucleotide is complementary to the genomic DNA located in the nucleolar region of the nucleus. This rDNA is transcribed into 18S ribosomal RNA.

The location of the transfected semiconductor (in this case titanium) was mapped by detecting titanium-specific K_{α} X-ray fluorescence induced at the—ID-E X-ray beamline at Sector 2 of the Advanced Photon Source at Argonne National Laboratory, Argonne, Ill.

Depending on the type of experiment 20 to 50 percent of the cells accepted and retained titanium nanoparticles. The addition of free oligonucleotides generally increased the success of titanium nanocomposite transfection and retention. As such, the free oligonucleotides serves as a means to facilitate transport to the targeted region in the cell.

FIG. 6 shows signals of titanium and phosphorous overlapped with each other. Panel C of FIG. 6 shows that the titanium signal is highest in the circular subregion of the nucleus where the nucleolus is situated. Retention within the nucleolus is dependent on hybridization/annealing of the R18Ss-Ti nanocomposite with the genomic ribosomal 18S rDNA.

The following applications can be envisioned:

Sensors of DNA-protein

Complex Formation Detail.

In this instance, the titania-dopamine-oligo construct is attached to DNA which is capable of hole scavenging (electron donating). Upon illumination of the construct, electrons are transferred from DNA to the valence band of TiO₂ to neutralize the positive holes originally produced from the photogenerated electrons which resided there (and which now reside in the conductance band of the titania). This changes the optical properties of TiO₂ and a blue color (absorption at ˜600 nm) appears. Subsequent addition of a source of conductive metal ions such-as silver, gold or copper ions will reduce metal ions to form conductive elemental metal. Upon DNA binding to electron-withdrawing proteins, the electron transfer does not occur and the change of the optical properties does not occur. As such, a lack of color change or lack of alteration of the electronic state of the titania particle indicates that an electrophillic moiety is attached to the titania-dopamine-oligo construct.

In another embodiment, the invention is carried out by applying free standing DNA and PNA oligo and peptide modified (1-20 nm) semiconductor nanocrystallites such as TiO₂ in solution. The modifier such as DNA or PNA oligo provides both selective hybridizing and hole scavenging functions (photochemical response) in vivo. The particles will bypass cell, nuclear, or mitochondrial membranes using standard delivery methods combined with attachment to peptides that facilitate entry into cells or their compartments.

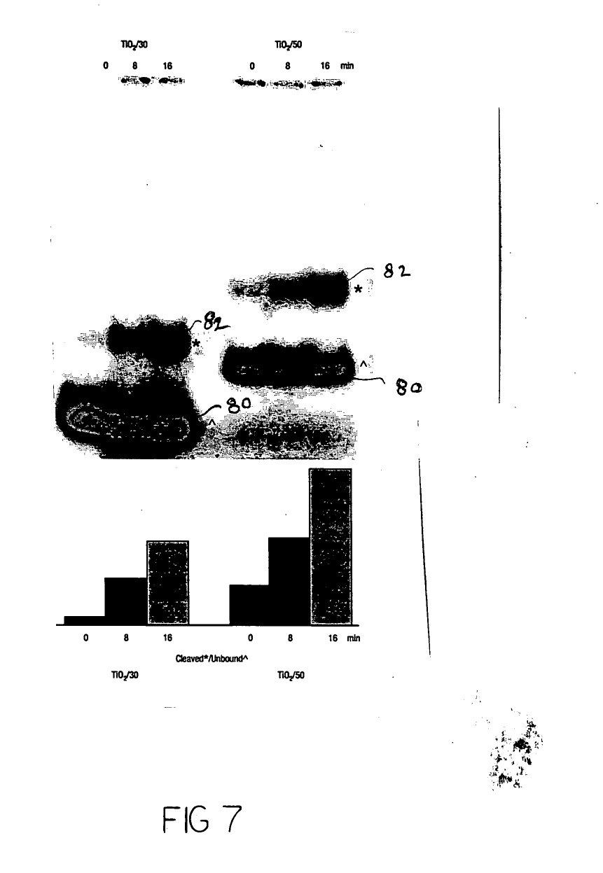

FIG. 7 depicts the characteristic of the invented construct as a light-inducible nucleic acid endonuclease. FIG. 7 is a photograph of polyacrylamide gel electrophoresis comparing mobility of single stranded, labeled DNA 80 and double stranded labeled DNA 82. The first three lanes are annealed nanocomposite TiO₂/30; the second three lanes are annealed nanocomposite TiO₂/50. Reaction mixtures were illuminated for 0, 8, or 16 minutes and loaded on the gel as indicated. The same labels apply to the bar graph below the gel photograph. Bar values correspond to values obtained by dividing the signal intensity of the double-stranded DNA (resulting from light-induced DNA cleavage) with the signal intensity of the radio labeled single-stranded DNA.

The double stranded DNA molecules were the result of annealing between the invented construct and complementary DNA which contained a label. However, inasmuch as nanocomposites do not enter agarose, the appearance of the double-stranded DNA band occurs only when the nanocomposite is absent from the double strand. The nanoparticle was separated from the double stranded molecule only when the invented construct (previously hybridized with its labeled complementary DNA sequence) is subjected to radiation. That large bands of double stranded DNA are evident in that portion of the agarose exposed to 16 minutes of light, versus 0 minutes or 8 minutes, indicates that cleavage of the double strand DNA occurs from the inorganic particle in the presence of radiation.

The inventors also discovered that the invented inorganic-organic construct participates in polymerase chain reaction processes. Subsequent illumination of the completed PCR products containing the inorganic particles (e.g. TiO₂ nanoparticles) induced DNA strand breaks within a short distance from the particles.

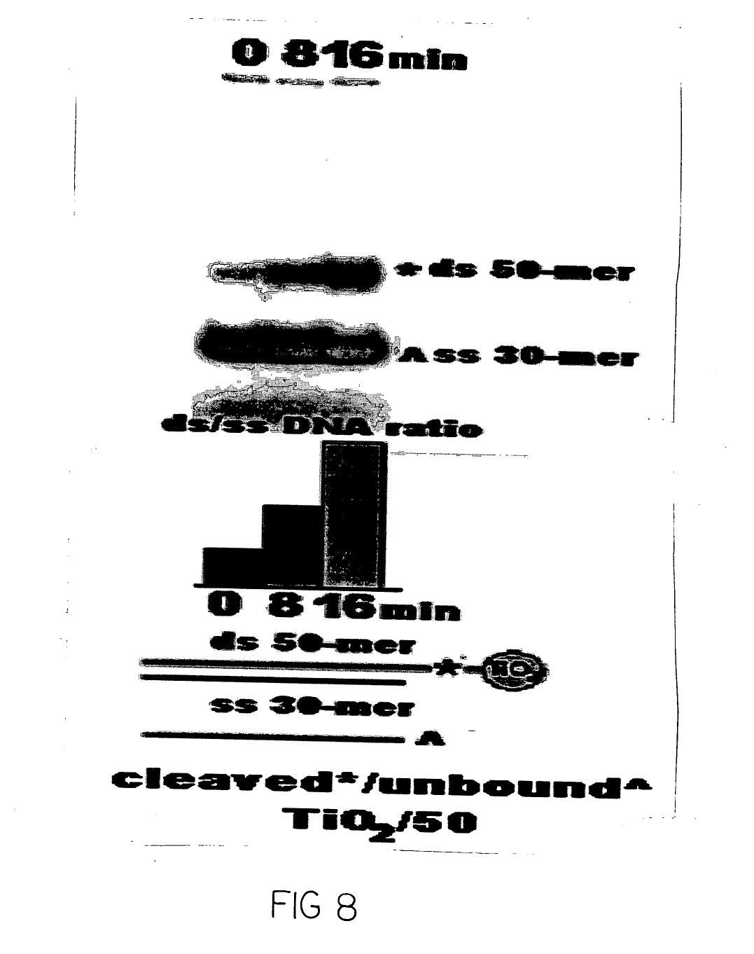

FIG. 8 depicts additional data depicting genetic manipulation using semi-conductor particles when subjected to light. FIG. 8 shows that the longer exposure to light, the more holes accumulate in the attached DNA, resulting in DNA cleavage. Wells in the gel in FIG. 8 contain a combination of TiO₂/50 nanocomposite annealed to a radio labeled complementary distal 30-mer illuminated 0, 8, or 16 minutes. A * marks the position of the double-stranded DNA, while a \hat{ } marks the position of the single-stranded complementary oligonucleotide.

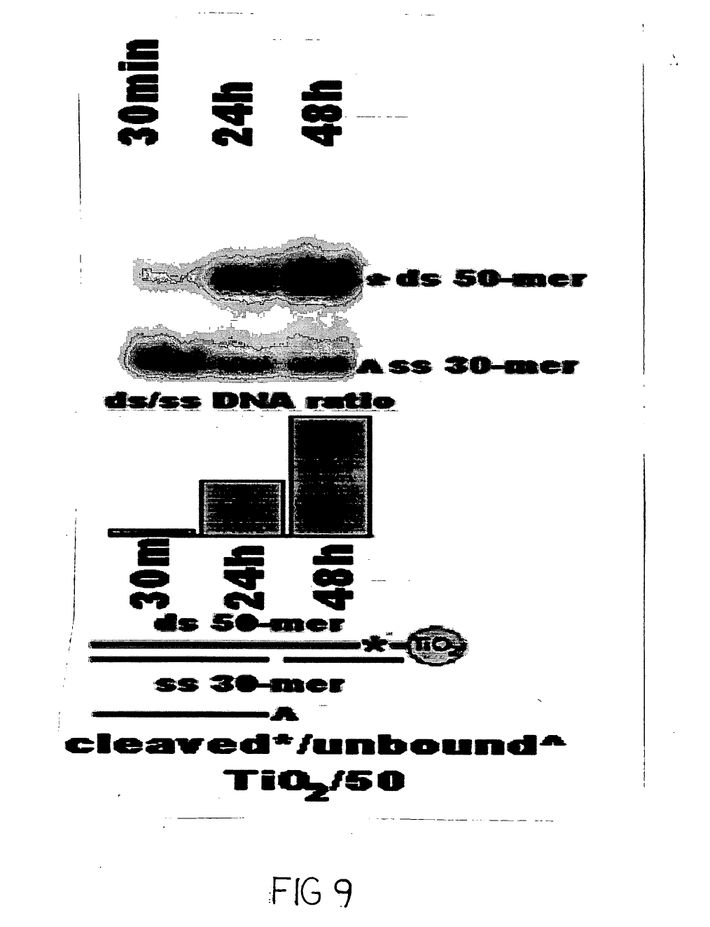

FIG. 9 depicts cleavage of nucleic acid molecules from semiconductor particles when the construct is incubated with radioactive labels. Specifically, incubation of nanocomposites with the radio labeled complementary oligonucleotide (so labeled by kinasing with γ[³²P] ATP in a cis position) induces cleavage of the DNA from a TiO₂-DNA composite. Length of exposure to radioactivity is indicated above the wells in the figure.

The following applications can be envisioned:

Gene Replacement

Particles are attached to peptides that facilitate entry into cells (and/or their compartments), and then to both a DNA strand with a copy of normal gene and a single stranded DNA or PNA oligo of sequence complementary to the host sequence where the excision is needed. Cutting will be achieved by oxidative damage produced by photogenerated holes at the TiO₂ surface. The resulting DNA break will facilitate recombination which will exchange the mutant/abnormal gene of the host with the introduced normal gene bound to the TiO₂ particle.

Gene Ablation

Particles are attached to peptides that facilitate entry into cells (and/or subcellular compartments), and then to a DNA strand with a single stranded DNA or PNA oligo of sequence complementary to the host sequence which is to be excised. Cleavage/excision are achieved by oxidative damage produced by photogenerated holes at the TiO2 surface. Resulting DNA breaks within amplified or abnormal genes will facilitate recombination which will excise excess copies or mutant copies of the gene.

Suicide Gene

Implantation Detail

Particles are attached to peptides that facilitate entry into cells (and/or their subcellular compartments), and then to both a DNA strand with a copy of a cellular suicide gene and a single stranded DNA or PNA oligo of sequence complementary to the host sequence of a promoter of a gene involved in proliferation or some other essential cellular function which is to be replaced to permit expression of the suicide gene. Excision will be achieved by oxidative damage produced by photogenerated holes at the TiO2 surface. The resulting DNA break will facilitate recombination which will exchange the host proliferative (or some other expressed) gene with the introduced suicide gene bound to the TiO2 particle.

In Vivo

Manipulation Detail

In another embodiment the invention is carried out by applying free standing oligonucleotide, PNA oligo, peptide or protein modified (1-20 nm) semiconductor nanocrystallites such as TiO₂ in solution. The modifier such as oligonucleotide, PNA or protein provide selective binding and hybridizing in vivo. The particles will bypass cell or subcellular membranes using standard delivery methods combined with attachment to peptides that facilitate entry into cells/subcellular compartments. The following applications can be envisioned:

Nanocrystalline particles are attached to a test DNA strand and also modified, if necessary, with a peptide that is responsible for “homing” into the nucleus and a peptide that facilitates entry into cells. After incubation sufficient for nuclear proteins to bind DNA, methylation of DNA is achieved using external agents. Particles are isolated after cell lysis and DNA is purified by hybridization to a solid support (column) and footprints are developed.

Nanoprotein

Torpedoes

A battery of specific proteins that carry out DNA repair are attached to nanocrystalline particle and delivered to the cells that require repair (such as those damaged by chemotherapy, radiation exposure, etc. or those from people with repair deficiencies). Additionally particles are attached to peptides that facilitate entry into cells/subcellular compartments.

Intracellular Drug

Delivery Detail

Drug molecules can be attached to particles together with antibodies or other molecules that recognize specific cell types and peptides that facilitate entry into cells/subcellular compartments for targeted drug delivery.

In situ PCR

Detail

PCR primer(s) and subsequent nested primer(s) can be attached to particles that can help visualize the genomic/chromosomal location of a PCR reaction. Particles can also have attached antigen that can serve for subsequent isolation of PCR products via Ag-Ab reactions. Particles are also attached to peptides that facilitate their entry into cells/subcellular compartments.

The inventors have discovered that semiconductor particle-biological molecule constructs participate in enzymatic reactions. Specifically, the invented construct participates in such enzymatic reactions as polymerase chain reaction, such as Taq polymerase-mediated DNA synthesis.

In Vitro

Manipulation Detail

In another embodiment the invention is carried out by applying DNA and PNA oligo modified (1-20 nm) semiconductor nanocrystalline films, such as TiO₂ thin films, on glass mesh or some other support. The modifier (for example, an oligonucleotide) provides both hole scavenging (photochemical and photophysical response) and selective hybridization binding functions in vitro. Following application can be envisioned:

Purification of Sequence Specific DNA

Binding Proteins or Proteins that Repair DNA

Nanocrystalline titania films on glass mesh or other supports modified with DNA of defined sequence or with specific base or nucleotide damage will selectively bind proteins that recognize the sequence of DNA or recognize damaged DNA.

Purification of RNA-Binding Proteins Essential

In Splicing, RNA Processing, or RNA Stability

Nanocrystalline titania films on glass mesh or other supports modified with DNA or RNA of defined sequence will selectively bind proteins that recognize the sequence of RNA and participate in splicing, RNA processing, or RNA stability.

Recyclable” Template for Transcription Coupled

Translation or Transcription Coupled Repair

Nanocrystalline titania films on glass mesh or other supports modified with DNA of defined sequence can serve as templates for in vitro transcription and translation. Similarly, nanocrystalline titania films or glass mesh of other supports modified with DNA that is damaged can serve as templates for in vitro repair of DNA or of transcription-coupled repair.

DNA Hybridization

Chip Detail

Oligonucleotides can be attached to titania films or other supports and their hybridization with probes can be detected via an electric signal created by an electron and hole migration in a miniature photoelectrochemical cell. The cell is created by immobilization of oligonucleotide modified TiO2 particles on conductive supports. Each of the particles modified with specific sequence would be connected into a separate circuit with a platinum or glassy carbon electrode immersed into an electrolyte containing an easily oxidizable redox couple such as absorbate or iodine. Another oxidizable redox couple is N,N,N′N′-Tetramethyl-p-phenylenediamine. Upon illumination holes created in the valence band of TiO2 will migrate to DNA chain provided that it is double stranded and an electron will be left on TiO2 and driven to platinum or glassy carbon cathode. Double stranded DNA is made only upon hybridization with probe. Selectivity (stringency) of hybridization can be adjusted to the thermal stability of each individual oligonucleotide and selectively probed at different washing temperature shifts. A very important feature of this type of “chip” is that it can be used both for mutation detection and monitoring of cDNA populations (gene expression).

DNA-Protein Interaction DNA Chip

Double stranded oligonucleotides can be attached to titania films or other supports in the above-mentioned photoelectrochemical cell. In this case all the leads will carry electrical impulses upon illumination. Upon coupling of double-stranded oligonucleotide with sequence specific proteins charge separation will be prevented. Therefore the lack of electrical signal would indicate DNA protein interaction.

Particle-Molecule

Preparation Detail

All the chemicals were reagent grade and used without further purification (Sigma, Aldrich or Baker). Triply distilled water was used. The pH was adjusted to pH 3.5 with NaOH or HCl. Oxygen was removed by bubbling with argon or nitrogen. Colloidal TiO₂ was prepared by dropwise addition of titanium(IV) chloride to cooled water. The temperature and rate of component mixing of reactants were controlled by an apparatus developed for automatic colloid preparation. One such apparatus is M. C. Thurnauer, Acta Scandinavica 51, pp. 610, (1997), and incorporated herein by reference.

The concentration of TiO₂ (0.1-0.6 M) was determined from the concentration of the peroxide complex obtained after dissolving the colloid in concentrated H₂SO₄, as described in Thompson, Inorganic Chem., 23, p 1794, (1989) and incorporated herein by reference. Surface modification of TiO₂ with ascorbate resulted in the charge transfer complex with optical properties which were described in T. Rajh, J. Phys. Chem. B., 103, pp 3513, (1999), and also incorporated herein by reference. Dopamine was also used for surface modification of TiO₂ but the onset of absorption in this system was further shifted to 810 nm.

The following specific protocol was utilized:

Dopamine was added into TiO₂ colloidal solutions at 8<pH<2.5. Immediate development of red color indicates instantaneous formation of the charge transfer complex between dopamine and TiO₂. The amount of adsorbed dopamine can be determined by measuring absorption at 440 nm, at extinction coefficient of 3.3×10³ M⁻¹cm⁻¹, absorption at 520 nm at an extinction coefficient of 1.1×10³ M⁻¹ cm⁻¹, or in higher concentrations with absorption at 570 nm with an extinction coefficient of 1×10² M⁻¹ cm⁻¹.

Dopamine/TiO₂ complex is extremely stable and cannot be removed by dialysis. The stability of the complex is larger than stability of the complexes between TiO₂ and glycidil, glycidil isopropyl ether, amino propyl silane or phenyl silane. At alkaline pH>8.5 dopamine modifier is readily removed from the TiO₂ surface.

Coupling of dopamine end-labeled oligonucleotides to TiO₂ particles should be performed at pH 6.5 and in 10-40 mM phosphate buffer. TiO₂ colloids prepared at pH 3.5 were diluted to 0.015M and 50 ml was mixed with 100 μl of glycidil isopropyl ether. With vigorous mixing, 1 ml of 0.2 M LiOH was rapidly injected into the TiO₂ solution until a pH of 9.5 was reached, per the protocol disclosed in Rajh, T. Langmuir, 8, 1265 (1992). The solution is dialyzed against 10-40 mM phosphate buffer until it reaches pH 6.5.

Binding end-labeled DNA with dopamine to TiO₂ results in the same optical changes and therefore could be quantified in the same manner.

Transient absorption measurements were performed at room temperature on the system previously described in Greenfield. S. R., Opt. Lett. 20, p 1394 (1995), and incorporated herein by reference.

EPR: The direct detection time-resolved X-band EPR experiments were collected on a Bruker ESP300E spectrometer equipped with a Varian cavity and a variable temperature cryostat (LTD) cooled to helium temperatures. The transient signal at 1 μs after laser flash and a dark background signal between the laser pulses were collected with Stanford Research System gate integrators and boxcar averagers.

Samples were excited using a nanosecond OPO laser system (Opotek Inc.). Light modulation-field modulation X-band EPR experiments were collected on a Varian E-9 EPR spectrometer equipped with a Varian cavity and a variable temperature cryostat (Oxford) cooled at helium temperatures. The light modulation experiments were carried out as described in L. L. Feezel et al. Biochim Biophy. Acta 974 p. 149 (1989). Xe 300 W lamp (Orion Corp) pulsed at 500 Hz was used as an excitation source.

Samples were checked for background EPR signals before irradiation. The g-factors were calibrated by comparison to a Mn²⁺ standard in SrO matrix (g=2.0012±0.0002), as described in J. Rosenthal, et al., Rev. Sci. Inst., 37, 381 (1966) and incorporated herein by reference.

In summary, hybrid complexes of semiconductor particles and biological molecules have been invented to target specific DNA or RNA sequences, and cleave them within 20 nucleotides from the annealing site, the annealing site being the interface between the particle and the biological molecule. This is achieved through the combined use of the semiconductor properties of TiO2 and the biological selectivity (molecular recognition such as by DNA-DNA hybridization) of the attached biopolmers.

In operation, the inventors have selectively bound hybrid tiO2-oligo to DNA in a cell. Once the TiO2 is linked to DNA, that DNA is cleaved by using light or radioactive probes. This facilitates the introduction of TiO2-oligo constructs (whereby the oligo is sequence specific for a target genome site) into genomes targeted for manipulation. Upon persistent but mild “irradiation” of the organelle, cell, or nucleus in which the construct resides, the gene sequence responsible for the characteristic will be cleaved. This results in cessation of the production of the protein responsible for the expression of that characteristic. As noted supra, details for this manipulation are found in Paunesku, et al., previously incorporated herein by reference.

While the invention has been described with reference to details of the illustrated embodiment, these details are not intended to limit the scope of the invention as defined in the appended claims.

1. A method for detecting molecules, the method comprising:
a) determining the electronic status of a semi-conductor;
b) establishing electronic communication between the molecules and the semiconductor;
c) subjecting the semi-conductor to energy influx;
d) redetermining the electronic status of the semi-conductor.

2-27. (Canceled)

28. A method for manipulating biological material in vivo, the method comprising:
a) attaching a semi-conductor to a first biological moiety to create a construct;
b) inserting the construct into a living organism;
c) allowing the construct to migrate to the biological material;
d) creating a plurality of charges on the construct, wherein the size of the charges and distances between the charges cause the biological material to change in structure.

29. The method as recited in claim 28 wherein the biological material comprises molecules selected from the group consisting of nucleotides, nitrogenous heterocyclic bases, amino acids, and combinations thereof.

30. The method as recited in claim 28 wherein the charges are created by subjecting the construct to radiation.

31. The method as recited in claim 30 wherein the radiation has an energy greater than 1.6 eV.

32. The method as recited in claim 28 wherein the radiation has energy ranging from about 1.6 eV to 10 eV.

33. The method as recited in claim 28 wherein the step of creating a plurality of charges further comprises subjecting the construct to radiation selected from the group consisting of white light, ultra violet light, X-rays or gamma rays, alpha rays, gamma rays, and combinations thereof.

34. The method as recited in claim 28 wherein the biological material is nucleic acid and the construct changes the nucleic acid by cleaving it.

35. The method as recited in claim 34 wherein the cleavage occurs when the semiconductor accumulates electrons from the first biological moiety.

36. The method as recited in claim 28 wherein the semiconductor is a metal oxide selected from the group consisting of TiO₂, ZrO₂, VO₂, MnO₂, NiO, ZnO, CuO, FeO₄ and combinations thereof.

37. The method as recited in 1 wherein the biological molecule is nucleic acid having base sequences interspersed with guanine.

38. The method as recited in claim 30 wherein the source of radiation is a radioactive isotope selected from the group consisting of phosphorus-32, iodine-123, iodine-131, sulfur-35, selenium-75, technetium-99, yttrium-90 and combinations thereof.

39. The method as recited in claim 37 wherein the radioactive isotope is covalently attached to the semi-conductor.


SEQUENCE LISTING (6 sequences)

SEQ ID NO: 1 - DNA, 16 bp, artificial sequence
  synthetic construct
ngcatgcatggatgga

SEQ ID NO: 2 - DNA, 16 bp, artificial sequence
  synthetic construct
nggatggatggatgga

SEQ ID NO: 3 - DNA, 16 bp, artificial sequence
  synthetic construct
nccactttccacacag

SEQ ID NO: 4 - DNA, 16 bp, artificial sequence
  synthetic construct
nagaccaagagccttc

SEQ ID NO: 5 - DNA, 16 bp, artificial sequence
  synthetic construct
nttccttggatgtggt

SEQ ID NO: 6 - DNA, 16 bp, artificial sequence
  synthetic construct
ncaggattccctcagt